(19) United States
(12) Patent Application Publication
(10) Pub. No.: US 2004/0071701 A1
(43) Pub. Date: Apr. 15, 2004

(54) Use of NGF-antagonists for the prevention or treatment of chronic visceral pain

(76) Inventors: Laure Delafoy, Antony (FR); Laurent Diop, Saclay (FR)

Correspondence Address: PFIZER INC., PATENT DEPARTMENT, MS8260-1611, EASTERN POINT ROAD, GROTON, CT, 06340, US

(21) Appl. No.: 10/269,285

(22) Filed: Oct. 11, 2002

(51) Int. Cl. A61K 39/395

(52) U.S. Cl. 424/145.1

(57) ABSTRACT

Use of a nerve growth factor (NGF) antagonist for the manufacture of a medicament intended for the prevention or treatment of chronic visceral pain and corresponding pharmaceutical compositions.

### FIELD OF THE INVENTION

The present invention relates to the use of NGF antagonists for the prevention or treatment of chronic visceral pain, such as chronic visceral pain due to a physiological disorder, for example dysmenorrhoea, dyspepsia, gastrooesophageal reflux, pancreatitis, visceralgia or irritable bowel syndrome.

### TECHNOLOGICAL BACKGROUND

There are two general categories of medicaments for the treatment of pain, both of which have disadvantages:

(1) nonsteroidal anti-inflammatory therapeutic compounds which are used to treat mild pain, but whose therapeutic use in the visceral sphere is limited by undesirable gastrointestinal effects such as gastric erosion, the formation of peptic ulcer or the inflammation of the duodenum and of the colon;

(2) morphine and related opioids, which are used to treat moderate to severe pain but whose therapeutic use is limited because of undesirable effects such as constipation, respiratory depression and the risk of addiction.

A need therefore exists for identifying compounds capable of bringing relief, with no side effects, to the patient suffering from chronic pain, and particularly chronic visceral pain.

Although the precise mechanisms for visceral pain differ depending on the organs and organ systems, two principles commonly apply to all types of visceral pain.

According to a first principle, the neurological mechanisms of visceral pain differ from those involved in somatic pain and thus the available experimental results concerning somatic pain cannot be extrapolated a priori to visceral pain.

According to a second principle, the perception of visceral pain by the patient and the psychological process to which they are subjected differ from those encountered in the case of somatic pain.

Among the types of visceral pain, it is possible to distinguish acute visceral pain and chronic visceral pain. In general, acute visceral pain is associated with an inflammatory situation and is in fact likened by persons skilled in the art to so-called inflammatory pain. The study of the physiology of acute visceral pain is thus carried out in an experimentally induced inflammatory situation.

It emerges from the above observations that the mechanisms involved in different physiopathological situations, such as acute visceral pain and chronic visceral pain, although unknown up until now, are distinct.

This is in addition confirmed by the fact that the classes of candidate therapeutic compounds for treating either type, acute or chronic, of visceral pain are different.

In the case of chronic visceral pain, the candidate therapeutic compounds suggested are in particular the following compounds:

(1) 5-HT antagonists which inhibit the binding of serotonin to the 5-HT-type receptors.

(2) Cholecystokin (CCK) antagonists.

(3) Opioid substances.

(4) Hypothalamic factors, such as analogues of somatostatin or analogues of gonadotrophin-releasing hormone.

Few medicaments are therefore known to act selectively on the hypersensitivity linked to gastrointestinal disorders (FARTHING M.J., 1998, Drugs, vol. 56: 11-21).

### SUMMARY OF THE INVENTION

The inventors focused on finding compounds capable of bringing relief to the patient suffering from chronic visceral pain and therefore of acting on at least one of the targets physiologically involved in the manifestation of these types of chronic visceral pain, which targets were unknown before the invention.

One aspect of the invention consists in using the capacity of NGF-antagonists to bring relief to the patient suffering from chronic visceral pain.

It has been shown, in accordance with the invention, that nerve growth factor (NGF) antagonists were capable of inhibiting or blocking the visceral hypersensitivity present in the pathophysiology of visceral functional disorders, in the case of chronic pain.

According to the invention, the expression chronic visceral functional disorders is understood to mean disorders of the sensitivity of the viscera having a nervous origin, also known by the name visceralgia. The viscera include the digestive, respiratory and urogenital organs and the endocrine systems, as well as the spleen, the heart and the large vessels.

From the medical point of view, a chronic visceralgia is characterized by a threshold of sensitivity to pain which is lowered compared with the normal threshold, in response to external mechanical stimuli.

Chronic visceral pain is in addition characterized by the absence of an inflammatory situation concomitant with the functional disorders.

For the purposes of the invention, chronic visceral pain includes the following chronic disorders:

chronic dyspepsia, a functional digestion disorder occurring in the absence of a detectable organic lesion and which may be symptomatic of other diseases or other disorders;

chronic dysmenorrhoea, characterized by pain associated with menstruation;

chronic pancreatitis, which is characterized by rapid loss of weight, asthenia, pain at the pancreatic point, a jaundice with distension of the gall bladder and digestive disorders due to pancreatic insufficiency, including hereditary chronic pancreatitis, a dominant autosomally transmitted disease which manifests itself from childhood by abdominal and recidivous painful attacks and which is characterized in adults by signs of insufficiency as well as by calcifications of the pancreas;

chronic gastrooesophageal reflux, which is characterized by a return into the oesophagus of the acidic gastric content and which causes, generally after a meal, ascending retrosternal burns, sometimes accompanied by acidic regurgitations;

IBS (irritable bowel syndrome), which is a non-inflammatory chronic disease characterized by abdominal pain and diarrhoea and/or constipation, with no detectable biochemical and histological modification.

The criteria for the diagnosis of IBS are (1) abdominal pain or discomfort which is relieved by defecation and which is associated with a modification of the frequency and of the consistency of the stools and (2) an irregular defecation profile characterized by at least three of the following phenomena: (a) frequency of the stools affected, (b) form of the stools altered, (c) passing of the stool affected, (d) passing of mucus, and (e) sensation of abdominal distension.

Chronic visceral pain, in particular gastrointestinal pain, is characterized by an abnormal perception of various external stimuli in the patients or in the animal. This abnormal perception of external stimuli may be defined as a decrease in the sensitivity threshold of the patient or of the animal to these external stimuli, compared with a control subject.

This physiopathological condition in which a stimulus which is not painful under normal conditions is perceived as being painful and which corresponds to a decrease in the sensitivity threshold is called allodynia.

It has thus been shown, according to the invention, that the administration of a nerve growth factor (NGF) antagonist to a subject suffering from chronic visceral pain made it possible to abolish the lowering of the sensitivity threshold of this subject to external stimuli, with a return to a sensitivity threshold comparable to that observed in a control healthy subject.

The subject of the present invention is therefore in particular the use of a nerve growth factor (NGF) antagonist for the manufacture of a medicament intended for the prevention or treatment of chronic visceral pain.

The invention relates in particular to the use of a nerve growth factor (NGF) antagonist which is binds to the said nerve growth factor.

The invention preferably relates to the use of a nerve growth factor (NGF) antagonist which is an antibody which binds specifically to the nerve growth factor (NGF).

The invention also relates to the use of a nerve growth factor (NGF) antagonist which binds to the Tyrosine kinase A nerve growth factor receptor.

The invention also consists in the use of a nerve growth factor (NGF) antagonist which binds either to NGF, or to the Tyrosine kinase A NGF receptor for the manufacture of a medicament intended for the prevention or treatment of chronic visceral pain due to a physiological disorder such as dysmenorrhoea, dyspepsia, gastrooesophageal reflux, pancreatitis, visceralgia and irritable bowel syndrome.

Another aspect of the invention is a pharmaceutical composition for the prevention or treatment of chronic visceral pain, characterized in that it comprises a pharmaceutically effective quantity of a nerve growth factor (NGF) antagonist, in combination with one or more pharmaceutically acceptable excipients.

A pharmaceutical composition according to the invention contains in particular a nerve growth factor (NGF) antagonist which binds to the said nerve growth factor.

A pharmaceutical composition according to the invention preferably contains a nerve growth factor (NGF) antagonist which is an antibody which specifically binds to the nerve growth factor (NGF).

Another pharmaceutical composition according to the invention contains a nerve growth factor (NGF) antagonist which binds to the Tyrosine kinase A which is an nerve growth factor receptor.

A pharmaceutical composition according to the invention is characterised in that it is intended for the prevention or treatment of chronic visceral pain due to a physiological disorder such as dysmenorrhoca, dyspepsia, gastrooesophageal reflux, pancreatitis, visceralgia and irritable bowel syndrome.

Preferably, a pharmaceutical composition according to the invention is formulated for oral administration.

### BRIEF DESCRIPTION OF THE FIGURES

FIG. 1 illustrates the effect of NGF injected intraperitoneally at various doses on the colonic pain threshold. The results are expressed as the mean plus or minus the standard error of the mean (SEM) of the pressure values. The test carried out is a two-sided Student's T test, of the unequal variance type with 2 examples. ns means not statistically significant, ** means p less than 0.01 and *** means p less than 0.001 versus control threshold.

FIG. 2 illustrates the effect of an anti-NGF antibody and an anti-TGFβ antibody, used as control antibody, on the colonic pain threshold in rats treated beforehand with TNBS (trinitrobenzenesulphonic acid). The results are expressed as the mean plus or minus the standard error of the mean (SEM) of the pressure values measured. The test carried out is a two-sided Student's T test, of the unequal variance type with 2 examples. *** means p less than 0.001 versus threshold of TNBS-treated rats receiving vehicle.

FIG. 3 illustrates the effect of ALE-0540 an NGF receptor antagonist on the colonic pain threshold in rats treated beforehand with TNBS (trinitrobenzenesulphonic acid). The results are expressed as the mean plus or minus the standard error of the mean (SEM) of the pressure values measured. The test carried out is a two-sided Student's T test, of the unequal variance type with 2 examples. ** means p less than 0.01, versus threshold of TNBS-treated rats receiving the ALE-0540 vehicle.

### DETAILED DESCRIPTION OF THE INVENTION

The expression “NGF antagonist” is understood to mean a compound capable of inhibiting the binding of nerve growth factor (NGF) to its receptor, Tyrosine kinase A (TrkA). That is to say:

a) The NGF antagonists according to the invention include compounds capable of specifically binding to NGF and of thus preventing its binding to the TrkA receptor.

b) Also forming part of the NGF-antagonists for the purposes of the invention, are the compounds capable of specifically binding to the TrkA receptor for NGF, thereby preventing the binding of NGF to its receptor.

A first group of antagonist compounds comprises antibodies which specifically bind either to the nerve growth factor (NGF), or to the TrkA receptor, such as those described in application PCT No. WO 97/21732, whose teaching is incorporated by reference into the present description.

In the case of an antibody specific for NGF, there may also be cited the purified anti-2.5S-NGF antiserum marketed by the company Sigma Chemicals (USA), in particular under the reference N-6655.

As regards the dose either of an antibody which specifically binds to NGF, or of an antibody which specifically binds to the TrkA receptor for NGF, this antibody will be preferably administered at the rate of 1 to 10 μg/kg of the weight of the patient per dose administered. This treatment of chronic visceral pain requires in general several successive administrations of the antibody, for example over time intervals ranging from one to four weeks.

The term patient is understood to mean a mammal and preferably humans.

The term “antibody” includes polyclonal and monoclonal antibodies, as well as antibody fractions, for example F(ab)′₂ or Fab, single chain antibody fragments (ScFv), chimeric antibodies or humanized antibodies.

A second group of antagonists of the invention comprises synthetic molecules.

By way of example, there may be mentioned the antagonists of neurotrophin described in application PCT No. WO 98/17278, peptides derived from NGF with antagonist effect, such as those described in application PCT No. WO 89/09225 and bicyclic peptides which are antagonists of NGF such as those described in application PCT No. WO 97/15593. The teaching of the various patents cited above is incorporated by reference into the present description.

Among these synthetic molecules are nerve growth factor (NGF) antagonists constituting a pharmaceutical composition according to the invention, which are chosen from the compounds binding to the said nerve growth factor.

The NGF-antagonists may also be compounds binding to the TrkA receptor for NGF.

The NGF-antagonists used according to the invention comprise solvates, hydrates and any pharmaceutically acceptable salts of such compounds.

The pharmaceutically acceptable salts of an NGF-antagonist which are used according to the invention comprise acetate, benzenesulphonate, benzoate, bitartrate, acetate of calcium, camsylate, carbonate, citrate, edetate, edisylate, estolate, esylate, fumarate, gluceptate, gluconate, glutamate, glycoloyl arsanilate, hexyl resorcinate, hydrabamine, hydrobromide, hydrochloride, hydrogen carbonate, as well as the other salts described in the review by BERGE et al. (1977, J. Pharm. sci., vol. 66: 1-19).

A pharmaceutical composition according to the invention is advantageously produced by formulating the NGF-antagonist in a dosage form comprising at least one pharmaceutically acceptable excipient or vehicle. To prepare a pharmaceutical composition according to the invention, the pharmaceutically acceptable vehicles may be either solids or liquids.

Preferably, a pharmaceutical composition according to the invention is characterized in that it is a formulation for oral administration.

Solid dosage forms for oral administration include gelatin capsules, tablets, pills, powders and granules.

In general, the pharmaceutically acceptable vehicles useful for the preparation of a composition for administration in vivo are in particular described in “REMINGTON's Pharmaceutical Sciences, 17th edition, Mack Publishing Company, Easton, Pa., 1985”.

Preferably, the nerve growth factor (NGF) antagonist is used for the manufacture of a medicament intended for the prevention or treatment of chronic visceral pain due to a physiological disorder such as dysmenorrhoea, dyspepsia, gastrooesophageal reflux, pancreatitis, visceralgia and IBS.

The subject of the invention is also a pharmaceutical composition for the prevention or treatment of chronic visceral pain, characterized in that it comprises a pharmaceutically effective quantity of a nerve growth factor (NGF) antagonist, where appropriate in combination with one or more pharmaceutically acceptable excipients.

The expression “pharmaceutically effective quantity” of a nerve growth factor antagonist is understood to mean, according to the invention, a quantity of the said antagonist compound which is capable of abolishing, in the subject considered, the decrease in the sensitivity threshold to external stimuli with a return of this sensitivity threshold to a level comparable to that observed in healthy subjects.

By way of illustration, a compound described as an antagonist of neurotrophins in publication PCT No. WO 98/17278 will be advantageously used in quantities allowing it to reach a concentration in the spinal fluid of between 1 and 500 μM.

In general, when it is not an antibody, an NGF-antagonist according to the invention will be administered at the rate of 0.1 to 300 mg/kg of the weight of the patient divided into one to three doses.

For an adult patient of normal weight, doses ranging from 5 to 500 mg per dose will be preferably administered.

The invention is in addition illustrated, without being limited as a result, in the following figures and examples.

### EXAMPLES

### A—Materials and Methods

A.1. Animals

Adult male rats of the Wistar strain, 320 to 350 g in weight (obtained from the Janvier farm, Le Genest-Saint-Isle, France) were used for all the experiments. They were kept under controlled conditions of temperature (20+/−1° C.), humidity (50+/−5%) and lighting (light from 7 to 19 hours). The animals were starved of food for 18 hours before the beginning of the experiments, the supply of water being maintained.

A.2. Behavioural Study

Distension studies were carried out on waking rats in isobaric mode, using pressure increments of 5 mm of mercury every thirty seconds. A latex balloon, placed in the distal part of the colon is linked to an electronic barostat. The pain threshold is defined as the pressure inducing the first abdominal contraction. Each rat is subjected to four distension trials so as to increase the reproducibility of the test. The mean of the pressure values is calculated on the thresholds observed for the four successive distensions.

A.3. Administration of TNBS

A laparotomy is carried out on rats anaesthetized with acepromazine (12 mg/kg i.p.) and ketamine (80 mg/kg i.p.) so as to inject into the proximal colon the trinitrobenzenesulphonic acid (TNBS) (50 mg/kg) in ethanol at 30%. The rats are then placed in individual cages. The colonic distension experiment is performed seven days after the administration of TNBS.

A.4. Administration of NGF, of Anti-NGF and Anti-TGFβ Antibodies

2.5 S-NGF obtained from mouse submaxillary gland (marketed by the company SIGMA under the reference N-6009) is dissolved in 0.1% bovine serum albumin (BSA). Into the naïve rats, 0.1 ng to 100 ng of NGF are injected by the i.p. route, 30 minutes before the distension.

The anti-NGF antibody marketed by the company SIGMA under the reference N-6655 is a fractionated rabbit antiserum directed against 2.5 S-NGF. The anti-NGF antibody, at the dilution of 1/2000 in sterile water, was injected by the i.p. route in a volume of 1 to 2 ml/kg, 30 minutes before the distension experiment.

The anti-TGFβ antibody (Anti-Pan Transforming growth factor) is the IgG fraction of an antiserum directed against the human TGFβ obtained in rabbits, marketed by the company SIGMA under the reference T-9429. The anti-TGFβ antibody, at a concentration of 9 μg/ml in sterile water was injected by the i.p. route in a volume of 2 ml/kg, 30 minutes before the distension experiment.

A.5. Administration of the NGF Receptor Antagonist, ALE-0540

The structure of the ALE-0540 compound is the following:

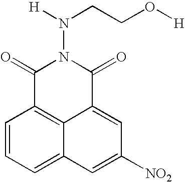

The NGF receptor antagonist, ALE-0540, at doses of 10 to 30 mg/kg was injected by the subcutaneous route in a volume of 2 ml/kg in cyclodextrin (20%, the ALE-0540 vehicle) in TNBS-treated rats, 30 minutes before the distension experiment.

### B. Results

### Example 1

### Effect of NGF on the Colonic Distension-induced Pain Threshold

Naïve rats were subjected to distension experiments with a balloon placed in the distal part of the colon. This is gradually inflated until an abdominal muscle reflex reaction is observed which reflects the onset of pain. The pressure applied to the balloon at the time of the abdominal muscle reflex determines the value of the colonic pain threshold.

The rats receive by the i.p. route either bovine serum albumin alone, or a solution of serum albumin containing 0.1 ng to 100 ng of NGF.

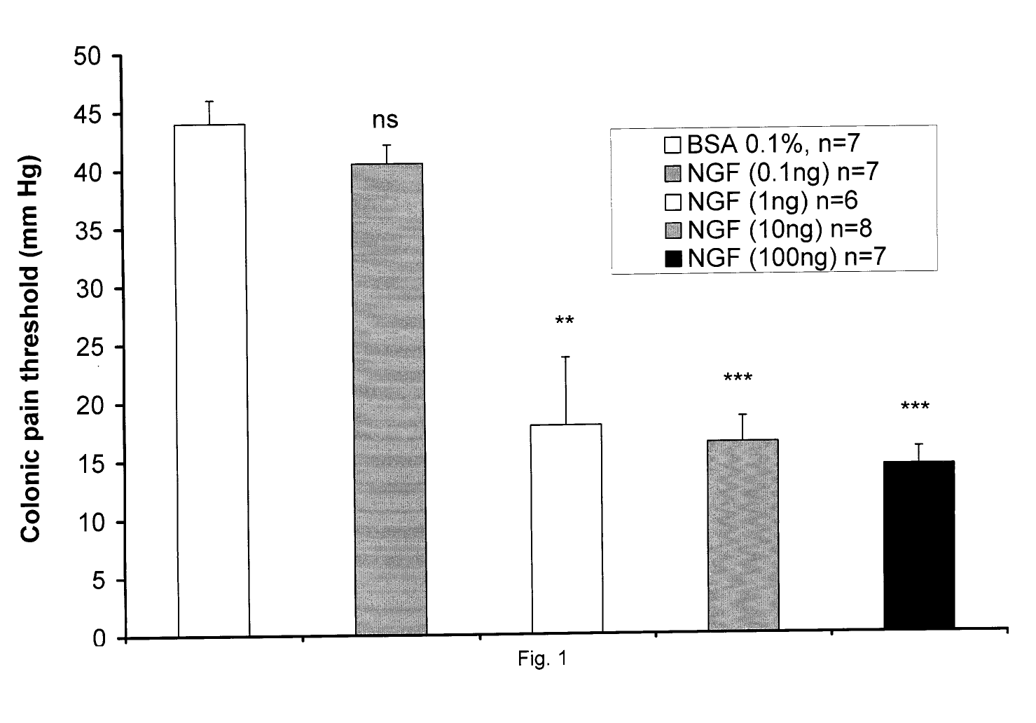

The results are represented in FIG. 1.

For the control rats which received only the bovine serum albumin, the colonic pain threshold corresponds to a pressure of about 44 mmHg (empty bar, to the left of FIG. 1).

It is possible to observe that increasing doses of NGF (0.1 ng to 100 ng) induce a significant reduction in the threshold of pain in the colon in the naïve rats (filled bars). Thus, the colonic pain threshold is lowered to less than 20 mmHg for 1 ng of NGF.

The experimental results of Example 1 therefore show that exogenous NGF induces visceral pain.

### Example 2

### Effect of an Anti-NGF Antibody on the Colonic Distension-induced Pain Threshold in TNBS-treated Rats

The induction of chronic allodynia in the colon was obtained by injecting TNBS into rats, seven days before the distension experiment, as indicated in the section Materials and Methods.

It was experimentally verified that no inflammatory-type situation is observed in the rats subjected to the experiment.

In particular, the level of activity of myeloperoxidase in the proximal colon collected from rats seven days after injection of TNBS made it possible to observe levels of myeloperoxidase activity of about 30 U/mg of proteins, whereas a level of activity of about 130 U/mg of proteins had been observed in proximal colon three days after the injection. Moreover, myeloperoxidase activity in the distal colon of TNBS-treated rats, three or seven days after the injection is not significantly different from myeloperoxidase activity in the distal colon of saline-treated rats.

1 U is the quantity of enzyme which determines an increase in the absorbence at 470 nm of 1.0 per minute at pH 7.0 and at 25° C., calculated relative to the initial rate with guaiacol as substrate.

The technique for measuring the myeloperoxidase activity used is that described by GRISHAM et al. (1990, Methods in enzymology, vol. 186: 729-742).

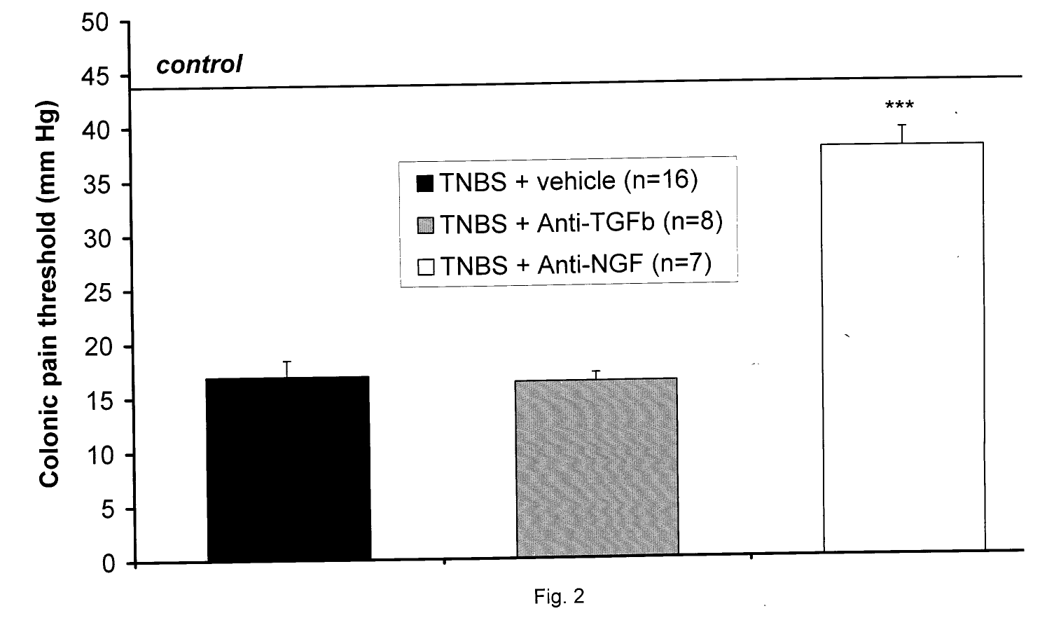

The results are presented in FIG. 2.

The control value for the threshold of sensitivity to pain in naïve rats is represented in the form of a line at about 44 mm of Hg.

The bars represent respectively from left to right:

(a) the mean value of the threshold of pain (+/−SEM) in rats treated with TNBS;

(b) the mean value of the threshold of pain (+/−SEM) in rats treated with TNBS, to which the anti-TGFβ antibody has been administered.

(c) the mean value of the threshold of pain (+/−SEM) in rats treated with TNBS, to which the anti-NGF antibody has been administered;

The colonic pain threshold in rats treated with TNBS is greatly reduced (about 17 mmHg) relative to the control rats (about 44 mmHg).

The administration of anti-NGF antibody (2 ml/kg at the dilution of 1/2000) reverses the effect of TNBS on the colonic pain threshold. Indeed, a pain threshold of 37.7+/−1.7 mmHg is obtained in the rats receiving the anti-NGF antibody against 16.9+/−1.5 mmHg for the rats treated with the vehicle. A p of less than 0.001 versus threshold of TNBS- and vehicle-treated rats was obtained by the Student's T test.

On the other hand, no modification in the colonic pain threshold is observed in TNBS-treated rats to which anti-TGFβ antibody has been administered.

This example clearly shows that an NGF-antagonist, such as the anti-NGF antibody used in these experiments, is capable of bringing the threshold of pain in the colon to a level comparable to that found in the control rats in which no chronic allodynia of the colon had been induced.

These results show that the action of NGF on the visceral sensory nerves contributes to the development of visceral hypersensitivity and that an NGF-antagonist is therapeutically effective in this type of specific digestive disorder and more generally in chronic visceral pain.

### Example 3

### Effect of ALE-0540, an NGF Receptor Antagonist, on TNBS-induced Colonic Allodynia in Response to Distension

ALE-0540 is a nonpeptidic nerve growth factor receptor antagonist.

The induction of chronic allodynia in the colon was obtained by injecting TNBS into rats, seven days before the distension experiment, as indicated in the section Materials and Methods.

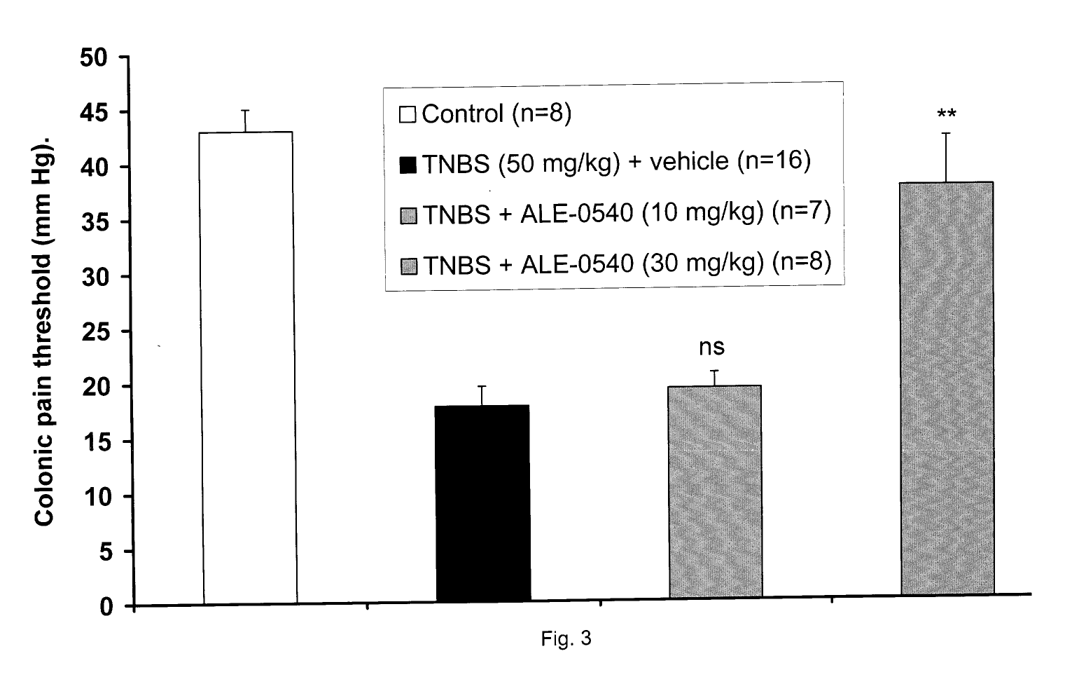

The results are presented in FIG. 3.

The control is the threshold value of pain in naive rats which is of about 44 mm of Hg.

The bars represent respectively from left to right:

(a) the mean value of the threshold of pain (+/−SEM) in rats treated with TNBS;

(b) the mean value of the threshold of pain (+/−SEM) in rats treated with TNBS, to which 10 mg/kg of ALE-0540, an NGF receptor antagonist has been administered;

(c) the mean value of the threshold of pain (+/−SEM) in rats treated with TNBS, to which 30 mg/kg of ALE-0540, an NGF receptor antagonist has been administered.

30 mg/kg ALE-0540 reverses the TNBS-induced colonic allodynia. Indeed, a colonic pain threshold of 37.6+/−4.5 mmHg is obtained in the TNBS-treated rats receiving 30 mg/kg ALE-0540 against 17.8+/−1.8 mmHg for the TNBS-treated rats receiving only the ALE-0540 vehicle.

A p of less than 0.01 versus TNBS-treated rats receiving vehicle was obtained for the Student's T test. Results are expressed as mean +/−SEM (n=7-8)

These results show that a non-peptidic NGF receptor antagonist exhibits antiallodynic activity in this model of visceral hypersensitivity.

1. Use of a nerve growth factor (NGF) antagonist for the manufacture of a medicament intended for the prevention or treatment of chronic visceral pain.

2. Use according to claim 1, characterized in that the nerve growth factor (NGF) antagonist binds to the said nerve growth factor.

3. Use according to claim 2, characterized in that the nerve growth factor (NGF) antagonist is an antibody which binds specifically to the nerve growth factor (NGF).

4. Use according to claim 1, characterized in that the nerve growth factor (NGF) antagonist binds to the Tyrosine kinase A nerve growth factor receptor.

5. Use according to claim 1 wherein the NGF antagonist is ALE-0540.

6. Use according to one of claims 1 to 5, characterized in that the medicament is intended for the prevention or treatment of chronic visceral pain due to a physiological disorder such as dysmenorrhoea, dyspepsia, gastrooesophageal reflux, pancreatitis, visceralgia and irritable bowel syndrome.

7. Pharmaceutical composition for the prevention or treatment of chronic visceral pain, characterized in that it comprises a pharmaceutically effective quantity of a nerve growth factor (NGF) antagonizing compound, in combination with one or more pharmaceutically acceptable excipients.

8. Pharmaceutical composition according to claim 7, characterized in that the nerve growth factor (NGF) antagonist binds to the said nerve growth factor.

9. Pharmaceutical composition according to claim 8, characterized in that the nerve growth factor (NGF) antagonist is an antibody which binds specifically to the nerve growth factor (NGF).

10. Pharmaceutical composition according to claim 7, characterized in that the nerve growth factor (NGF) antagonist binds to the Tyrosine kinase A which is a nerve growth factor receptor.

11. Pharmaceutical composition according to one of claims 6 to 10, characterized in that it is formulated for oral administration.

12. Method for the prevention or treatment of chronic visceral pain which comprises administering to a patient in need thereof a pharmaceutically effective quantity of a nerve growth factor antagonist.

13. A method according to claim 12 wherein the nerve growth factor (NGF) antagonist binds to the said nerve growth factor.

14. A method according to claim 12 wherein the nerve growth factor (NGF) antagonist is an antibody which binds specifically to the nerve growth factor (NGF).

15. A method according to claim 12 wherein the nerve growth factor (NGF) antagonist binds to the Tyrosine kinase A nerve growth factor receptor.

16. A method according to claim 12 wherein the nerve growth factor (NGF) antagonist is ALE-0540.

17. A method according to claim 12 wherein the chronic visceral pain is due to a physiological disorder selected from dysmenorrhoea, dyspepsia, gastrooesophageal reflux, pancreatitis, visceralgia and irritable bowel syndrome.
